(19) United States
(12) Patent Application Publication
(10) Pub. No.: US 2002/0123708 A1
(43) Pub. Date: Sep. 5, 2002

(54) Back board apparatus

(76) Inventors: J. Rickley Dumm, San Diego, CA (US); James Y.C. Sze, San Diego, CA (US)

Correspondence Address: Pillsbury Winthrop LLP, Intellectual Property Group, Suite 200, 11682 El Camino Real, San Diego, CA, 92130, US

(21) Appl. No.: 10/077,592

(22) Filed: Feb. 15, 2002

Related U.S. Application Data
(60) Provisional application No. 60/269,604, filed on Feb. 15, 2001.

(51) Int. Cl. A61F 5/00

(52) U.S. Cl. 602/32

(57) ABSTRACT

A back-stretching apparatus, capable of suspending a person comfortably in an upright position. The backboard comprises an inclined platform with footholds and a strap wrap. Facing away from the platform, a patient would step on to foothold and snugly wrap the strap wrap under the patient's arms, and across the patient's torso, securely fastening the patient. The patient may then step off the footholds leaving the patient freely and comfortably suspended. The platform may be fixed to a wall or free standing with the assistance of a support rack and a T-bar.

### RELATED APPLICATIONS

This application claims the benefit of co-pending U.S. Provisional Application Serial No. 60/269,604 filed Feb. 15, 2001.

### BACKGROUND

1. Field of the Invention

Aspects of the present invention relate in general to an apparatus to stretch the human spine through suspending a person comfortably in an upright position.

2. Description of the Related Art

Pressure from the compression of the spine may cause back pain for many individual sufferers. Back pain can be an incapacitating experience.

Various devices are available to stretch the human spine for therapeutic purposes. Many such devices hang patients upside down by their feet. For some people this is not a viable alternative. First, the hanging is uncomfortable as the upside-down patient gets dizzy or even experiences a headache from the experience. Second, the hanging and subsequent release requires at least another person to help.

### BRIEF DESCRIPTION OF THE DRAWINGS

FIGS. 1A and 1B illustrate the side view of a backboard embodiment to suspend a person comfortably in an upright position.

FIGS. 2A-2C illustrate the front, rear, and alternate embodiments of a platform to suspend a person comfortably in an upright position.

FIG. 3 illustrates a support rack embodiment to suspend a person comfortably in an upright position.

FIGS. 4A-4B illustrate a T-bar embodiment to support a structure to suspend a person comfortably in an upright position.

FIGS. 5A-5B illustrate an alternate T-bar embodiment to support a structure against a fixture, to suspend a person comfortably in an upright position.

FIGS. 6A-6B illustrate the side and front of an alternate embodiment of a platform to suspend a person comfortably in an upright position.

### DETAILED DESCRIPTION

What is needed is an easy-to-use apparatus capable of stretching the human spine through suspending a person comfortably in an upright position.

Aspects of the present invention include a wall-mountable and self-standing device to stretch the human spine through suspending a person comfortably in an upright position. These aspects include a freestanding backboard, and a backboard that may be fixed to a wall or other secure structure. The embodiments of the present invention enable patients to step on to a backboard foothold, strap themselves on an inclined backboard platform, and hang comfortably on the platform.

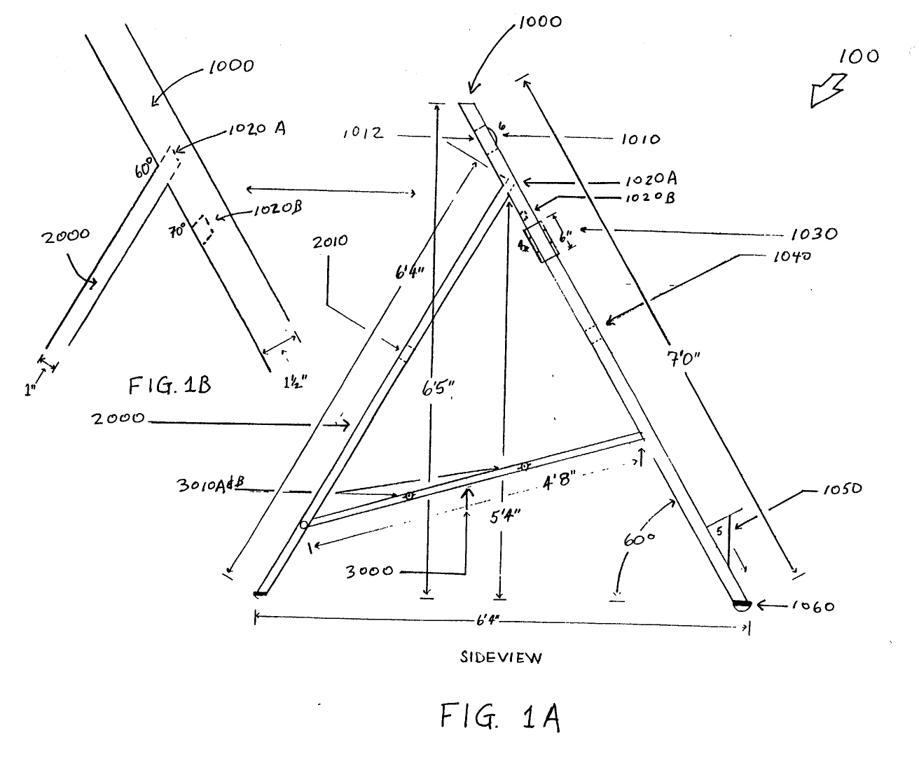

FIG. 1A illustrates a side view of a self-standing (or “freestanding”) backboard 100 embodiment, an apparatus to stretch the human spine through suspending a person comfortably in an upright position, constructed and operative with an embodiment of the present invention. As shown, backboard 100 comprises a platform 1000, support rack 2000, and a T-bar 3000. Platform 1000 supports the weight of a suspended patient. Support rack 2000 and T-bar 3000 enable platform 1000 to be freestanding in an inclined position, to suspend a patient. Platform 1000, support rack 2000, and T-bar 3000 may be constructed of wood, metal, fiber-glass, plastic, other composite materials, or any structural material known in the art with the elasticity, tensile and compressive strengths and toughness to support its own mass in addition to that of a suspended patient. In some embodiments, combinations of materials may be used, according to the design and use criteria. For example, in some portable embodiments, composite materials may be used to provide strong tensile strength while maintaining a light weight for portability.

The length and thickness of platform 1000 may vary depending upon the structural materials used in its construction. As shown in FIG. 1A, platform 1000 may be approximately 1.5 inches to 3 inches in thickness, and six feet or taller. The embodiment shown is seven feet in length. It is understood that these parameters of length and width are used for illustrative purposes only, and are not to be considered limiting. Embodiments of the invention are limited only by the claims. Platform 100 may include a headrest 1010, face-hole 1012, one or more support rack attachment points 1020A-B, a strapwrap 1030, hinges 1040, one or more footholds 1050, and skid-proof runner 1060. These structures and their use are described in greater detail below.

Support rack 2000 and T-bar 3000 may be any structures or structure known in the art capable of supporting platform 1000. As shown in FIG. 1, support rack 2000 may include hinged lock 2010. Similarly, T-bar 3000 may include latch locks 3010A-B. Both support rack 2000 and T-bar 3000 structures are discussed in greater detail below.

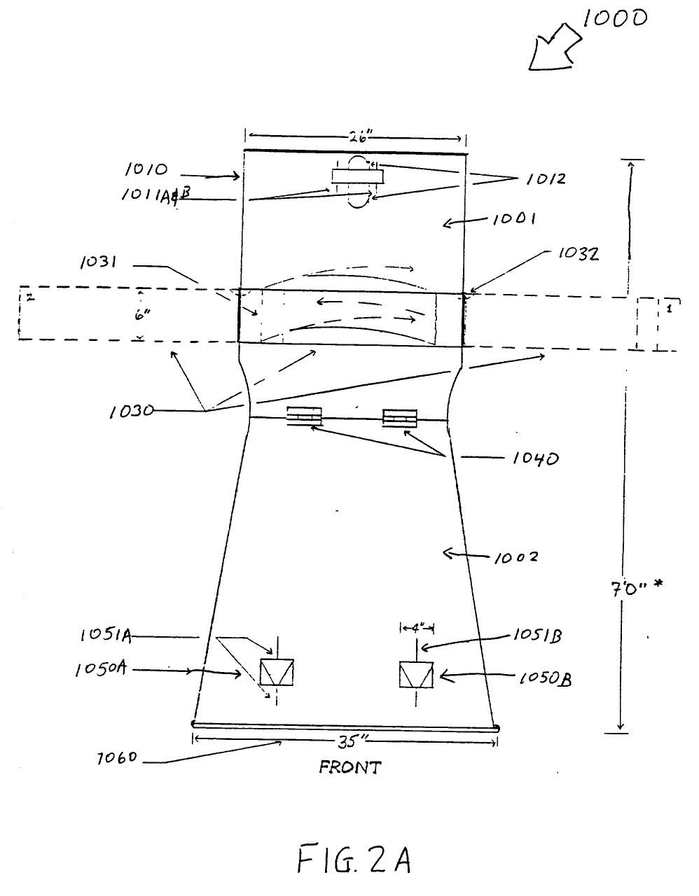

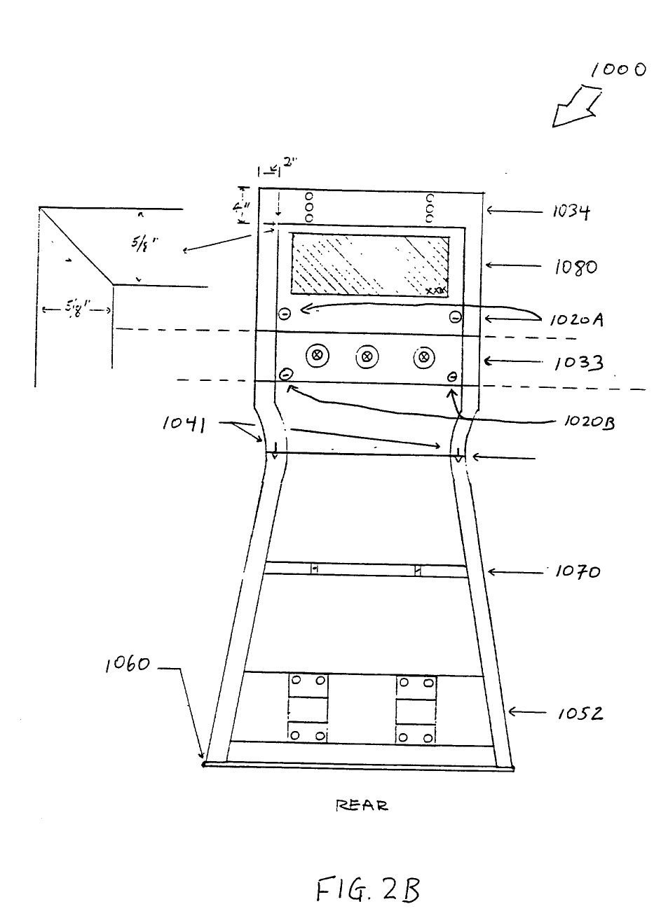

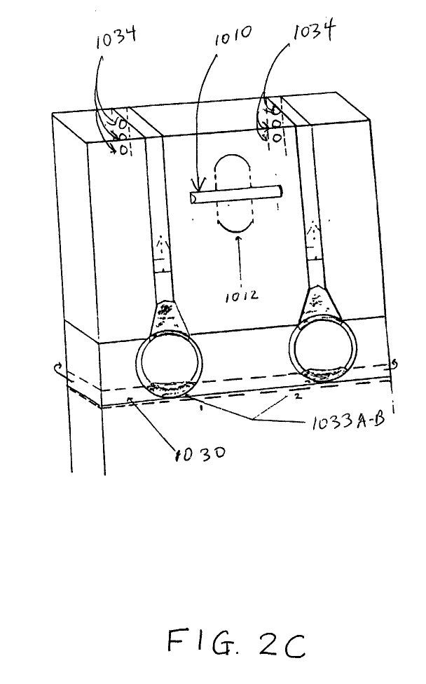

FIG. 2A illustrates the front view of an embodiment of platform 1000, to suspend a person comfortably in an upright position, constructed and operative with an embodiment of the present invention. Platform 1000 may be a “torso-like” shaped platform. As described above, headrest 1010, one or more support rack attachment points 1020A-B, a strapwrap 1030, hinges 1040, one or more footholds 1050, and skid-proof runner 1060.

In some foldable embodiments, platform 1000 may comprise two sections, a top section 1001 and a bottom section 1002. In such an embodiment, top section 1001 and bottom section 1002 may be joined by hinges 1040 recessed into the platform 1000 to allow platform 1000 to be flat and smooth. Hinges 1040 allow top section 1001 to be folded over bottom section 1002. In hinged embodiments, hinges 1040 may be secured in place by bolts and locked.

In some embodiments, headrest 1010 may be located in the center and on the upper-third of platform 1000. Headrest 1010 height may be adjusted along adjustment slides 1011A-B. Adjustment slides 1011A-B may be any structure known in the art to allow headrest 1010 to move and fix headrest 1010 height. In yet other embodiments headrest 1010 may be removable. Such embodiments, headrest 1010 may be fitted above a face-hole 1012. Face-hole 1012 is an opening through platform 1000 of sufficient size to allow a human face to be comfortably inserted.

Strap wrap 1030 is used to secure patient on to platform 1000, and may include fasteners 1031, such as Velcro™ strips, buttons, or any other fasteners known in the art. Strap wrap 1030 may also include padding 1032 to ensure secure support under the patient's arms. Strap wrap 1030 may require a fairly substantial width, approximately six inches, to distribute the patient's body weight securely while maintaining patient comfort. As shown in FIG. 2C, alternate embodiments of platform 1000, strap wrap 1030 may be used in conjunction with loop harness 1033A-B, constructed and operative with an embodiment of the present invention. Loop harness 1033 are two strap devices acting independently of each other. At one end snaps 1034, bolts, screws, or any other joining device known in the art secures the straps. At the other end of the strap 1033, a loop is provided to allow patients to slip an arm through. A loop is provided for each arm. The loops may be padded for under arm comfort. On the outer edges of the loops, fasteners 1031 may be added for use with strap wrap 1030. In some embodiments, a narrower strap wrap 1030 width may be used in conjunction with loop harness 1033. One example of a narrower strap wrap 1030 width would be two inches.

Returning to FIG. 2A, in some embodiments there may be one or more footholds 1050. Some embodiments may use a single wide (six or more inches) foothold 1050 placed at the center of platform 100. In embodiments that use two footholds (one for each patient foot), each foothold 1050A-B may be placed symmetrically off-center. Narrower (approximately four inch) footholds 1050 may be used in such embodiments. In some embodiments, the height of footholds 1050 may be adjusted along foothold track 1051. Foothold tracks 1051 may be any structure known in the art that allows a step to be vertically moved and secured.

A runner 1060 may cover the base of bottom section 1002 to prevent skidding. Such a runner may be made of rubber or any other high-friction surface known in the art. A wide base, such as approximately 35 inches, helps prevent platform 1000 from tipping.

Turning now to FIG. 2B, the rear of platform 1000 is illustrated, constructed and operative with an embodiment of the present invention. From the rear, platform 1000 may also include multiple support rack attachment points 1020A-B. Support rack attachment points 1020 are female inserts that allow support rack 2000 to “plug” into the attachment points 1020. In FIG. 2B, two sets of support rack attachment points 1020A-B are shown, to allow the platform 100 to be 60° and 70° from horizontal. It is understood that one or more sets of support rack attachment points 1020A-B may be placed to allow the platform to be held up to nearly 90° from horizontal. Additionally, support rack attachment points 1020 are depicted as being circular in shape, although they may be of any shape known in the art, as long as it corresponds properly to the shape of the cross section of support rack 2000.

FIG. 1B illustrates a side cross-section view of the joining of platform 1000 with of support rack 2000 via attachment points 1020A, constructed and operative with an embodiment of the present invention, constructed and operative with an embodiment of the present invention.

Returning to FIG. 2B, platform 1000 may also include a corresponding T-bar hitches 1070, similarly constructed with attachment points 1020.

In some embodiments, the hinges 1040 include snap-over locks 1041, which prevent platform 1000 from folding over in an incorrect direction.

As shown on the rear of platform 100, footholds 1050 may be secured to platform 1000 via bolts 1052, screws, nails, or any other similar structures known in the art. The bolts may be recessed in the rear platform surface in order to keep the surface flat and smooth.

In an alternate embodiment, platform 1000 may include a pouch 1080 to store optional harness 1033 and the like. Pouch 1080 may be constructed of any kind of flexible material known in the art, such as cloth or netting.

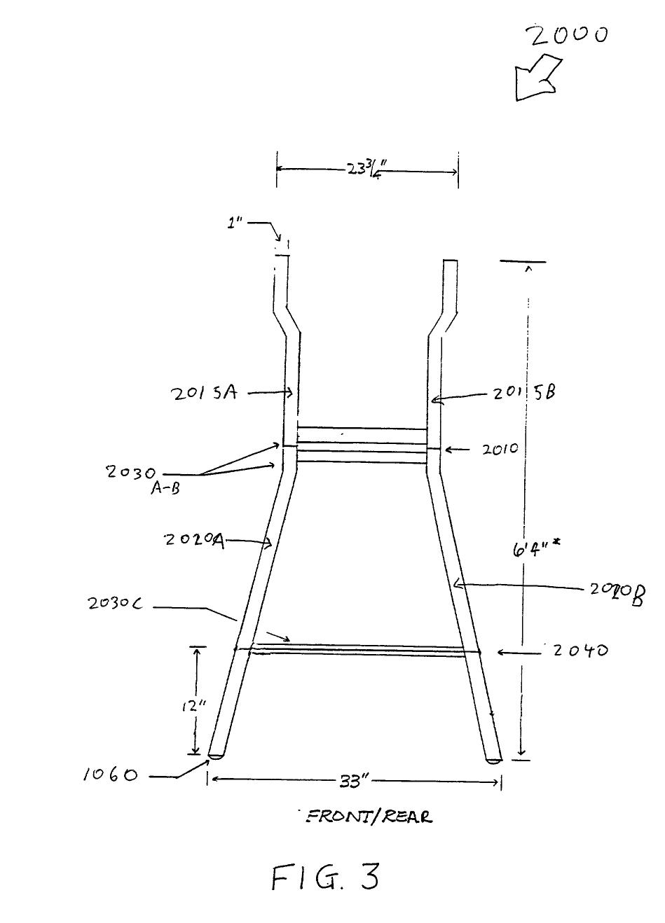

FIG. 3 illustrates a support rack 2000 embodiment, constructed and operative with an embodiment of the present invention. The support rack 2000 may be a lightweight, strong tubular stand, with installed structures. In some embodiments, the entire structure may be a single solid contiguous structure. The embodiment shown in FIG. 3, depicts a support rack 2000 adapted for portability, and thus folds upon itself using hinges 2010. In such an embodiment, support rack comprises two symmetrical upper frames 2015A-B and, two symmetrical lower frames 2020A-B. The two symmetrical upper frames 2015A-B are coupled by support bar 2030A. The symmetrical lower frames 2020A-B are coupled by two support bars 2030B-C. The materials used in the construction of the frames 2015A-B, 2020A-B, and 2060A-B are as described above. Support bar 2030C may rotate along an axis 2040.

Symmetrical lower frames 2060A-B may each have a runner 1060 to prevent skidding. Such a runner may be made of rubber or any other high-friction surface known in the art.

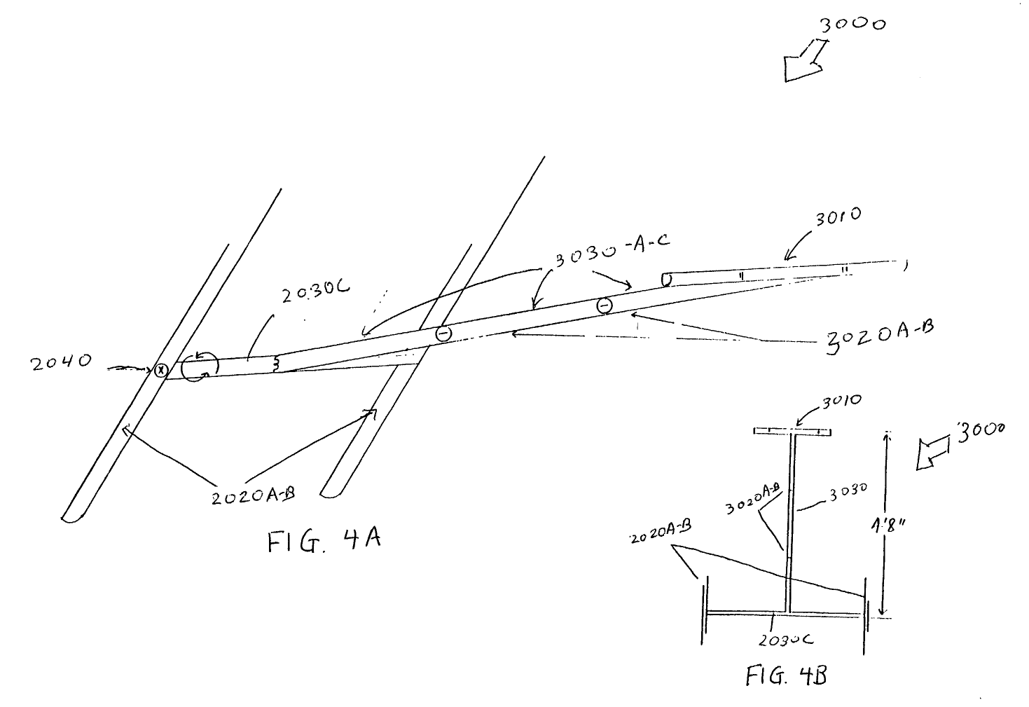

FIGS. 4A-4B illustrate a T-bar embodiment to support a structure to suspend a person comfortably in an upright position, constructed and operative with an embodiment of the present invention. FIG. 4A illustrates a side-view on how the T-bar may “accordion” in-and-out, locking and releasing against rack 2000, while FIG. 4B illustrates a fully extended T-bar embodiment from a top view. A T-bar comprises a platform bar 3010, a frame bars 3030A-C, and locks 3020A-B. Locks 3020A-B may be any structure as is known in the art to lock frame bars 3030A-C in a fixed position.

In an alternate embodiment, T-bar 3000 may be a one-piece rigid (non-foldable) structure, where frame bars 3030 and platform bar 3010 are a single “T-shaped” structure without requiring locks 3020A-B.

When accordioned out, the T-bar hooks onto the support bar 2030C on the support rack 2000.

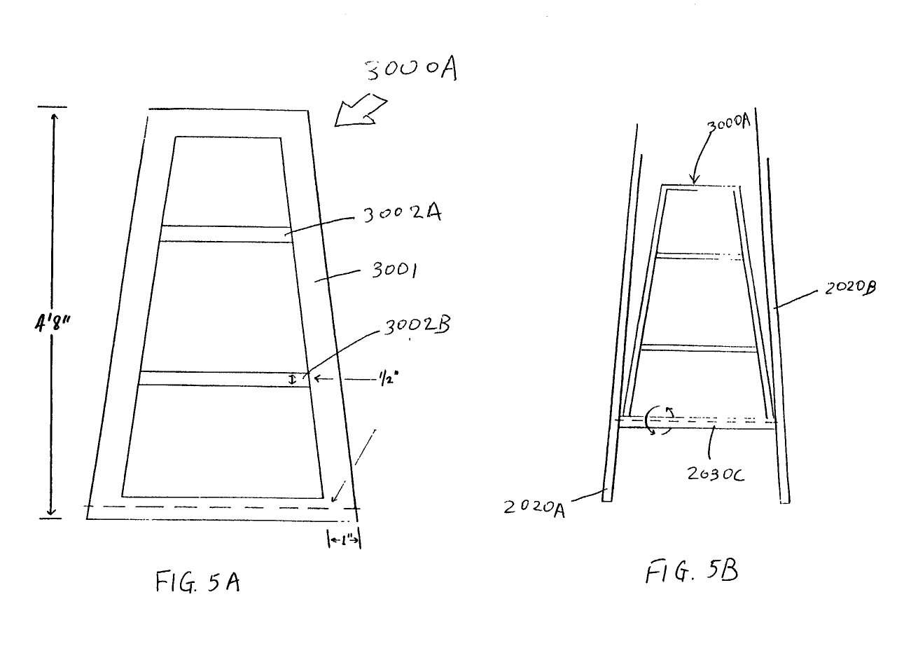

FIGS. 5A-5B illustrate another alternate T-bar embodiment to support a structure against a fixture, to suspend a person comfortably in an upright position, constructed and operative with an embodiment of the present invention. This figure illustrates that that T-bar embodiments may vary in size or shape, as long as the embodiment adequately supports platform 1000. FIG. 5A illustrates a top view of T-bar 3000A, while FIG. 5B illustrates T-bar 3000A embodiment connected with portions of support rack 2000. This embodiment of T-bar comprises an A-frame 3001 coupled with horizontally placed A-support members 3002A-B. As shown in FIG. 5B, the T-bar hooks onto the support bar 2030C on the support rack 2000.

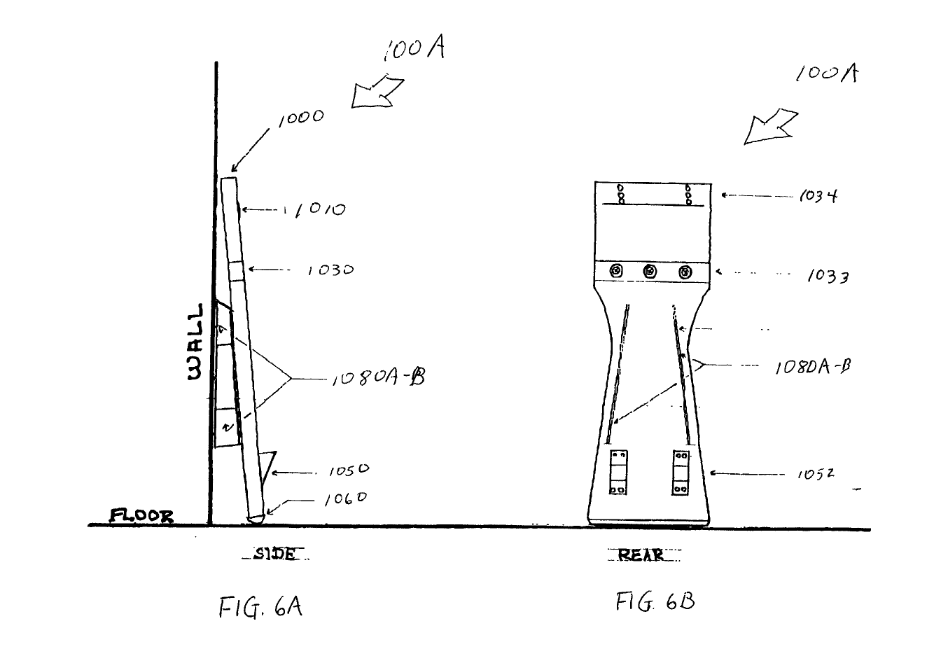

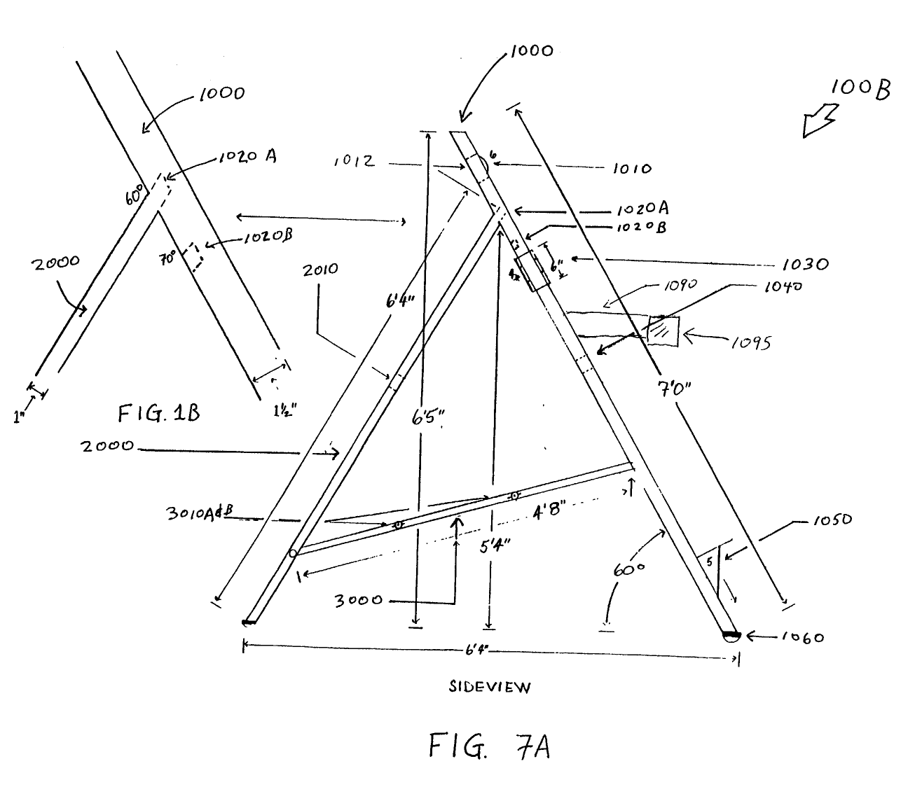

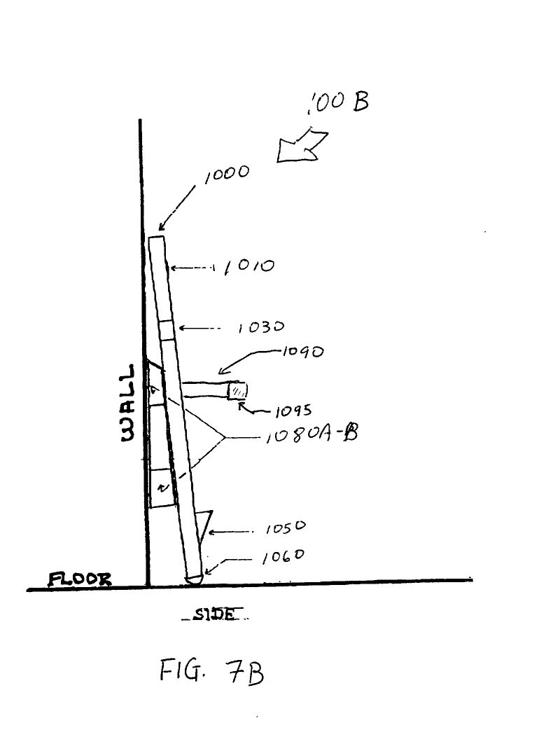

FIGS. 6A-6B illustrate the side and front of an alternate embodiment of a backboard 100 to suspend a person comfortably in an upright position, constructed and operative with an embodiment of the present invention. This embodiment of a backboard 100 comprises a platform 100 adapted to be securely fastened to a fixed structure, such as a wall.

FIG. 6A illustrates a side view of backboard 100A. Backboard 100A comprises platform 1000 and wedge-runners 1080. Platform 1000 may be identical to the embodiment described in FIGS. 2A-2C, configured to attach to wedge-runners 1080. Wedge-runners 1080 may be any structure known in the art to couple platform 1000 to a fixed structure such as a wall. Wedge-runner 1080 allows the platform 1000 to rest vertically at approximately 60-90°, the angle at which the patient will hang. In some alternate embodiments, platform 1000 will be a single structure, and not foldable.

In a foldable embodiment of the invention, the backboard 100 may be folded into a massage table, complete with face-hole 1012. Simply detaching the support rack 2000 from the platform, the support rack 2000 is folded into a triangular-legged stand, with the top-tips of two symmetrical upper frames 2015A-B resting on the floor. Platform 1000 is lowered until it rests on support bar 2030C, which may be foam padded. The resulting structure may be used as a massage table, sloping at approximately 200.

In some embodiments of, as pictured in FIGS. 7A-7B platform 1000 may further comprise arm rest 1090, and arm-rest fixture 1095, constructed and operative with an embodiment of the present invention. Arm rests 1090 may include attachable-detachable arm rests attached and secured in the platform 1000. Arm-rest fixture 1095 may include any fixture known in the art attachable to an arm-rest 1090, including, but not limited to, cup-holders, an attachable-detachable work tray or fold-out table, and pockets/trays for magazines, television remote-controls and the like.

Using an embodiment of the present invention, a patient would step on to foothold 1050, and face away from platform 1000. Snugly wrapping strap wrap 1030 under the patient's arms, and across the patient's torso, the fasteners 1031 are secured. Easily, one-by-one, the patient steps off the footholds 1050A-B, until the patient is freely and comfortably between the foothold.

To release a patient from the hanging position, the patient steps back on to the footholds 1050A-B, unfastens the fasteners 1031 of the strap wrap 1030, and steps off platform 1000.

The previous description of the embodiments is provided to enable any person skilled in the art to practice the invention. The various modifications to these embodiments will be readily apparent to those skilled in the art, and the generic principles defined herein may be applied to other embodiments without the use of inventive faculty. Thus, the present invention is not intended to be limited to the embodiments shown herein, but is to be accorded the widest scope consistent with the principles and novel features disclosed herein.

What is claimed is:

1. A back board apparatus to suspend a user in an upright position, comprising:
a platform with a top end and a bottom end;
a strapwrap, physically coupled to the platform, to suspend the user against the platform.

2. The apparatus of claim 1, further comprising:
a foothold step attached toward the bottom end of the platform.

3. The apparatus of claim 2, wherein the foothold step is adjustable in height.

4. The apparatus of claim 3, further comprising:
a head rest attached toward the top end of the platform

5. The apparatus of claim 4, wherein the head rest is adjustable in height.

6. The apparatus of claim 5, further comprising:
an anti-skid runner coupled to the bottom end of the platform.

7. The apparatus of claim 6, further comprising:
an arm rest coupled to the platform.

8. The apparatus of claim 7, the arm rest further comprising:
a cup holder configured to receive a beverage container.

9. The apparatus of claim 7, the arm rest further comprising:
a fold-out table configured to provide a flat level surface.

10. The apparatus of claim 7, the arm rest further comprising:
a magazine rack configured to store written material.

11. The apparatus of claim 6, further comprising:
a wedge runner to support the platform against a wall.

12. The apparatus of claim 6, further comprising:
a rack with a crossbar, configured to be coupled to the top end of the platform; and
a bar, configured to be coupled to the platform on one end, and configured to be coupled to the crossbar.

13. The apparatus of claim 12, wherein the top end and the bottom end are coupled by hinges.

14. The apparatus of claim 13, wherein the top end is shaped to form a hole near the headrest.

15. The apparatus of claim 14, further comprising:
a fastener configured to secure the strapwrap around the user.

16. The apparatus of claim 15, wherein the strapwrap is approximately six inches in width.

17. The apparatus of claim 16, further comprising:
two loop harnesses, coupled to the top end, configured to receive arms of the user.
